(19) United States
(12) Patent Application Publication
(10) Pub. No.: US 2021/0260148 A1
(43) Pub. Date: Aug. 26, 2021

(54) PEPTIDES FOR TREATING MUCOSA

(71) Applicant: Jean CARRUTHERS, Vancouvver (CA)

(72) Inventor: Jean CARRUTHERS, Vancouvver (CA)

(21) Appl. No.: 17/249,119

(22) Filed: Feb. 21, 2021

Related U.S. Application Data
(60) Provisional application No. 62/979,898, filed on Feb. 21, 2020.

(51) Int. Cl. A61K 38/06; A61K 38/05; A61K 38/07; A61K 47/24

(52) U.S. Cl. CPC A61K 38/06; A61K 47/24; A61K 38/07; A61K 38/05

(57) ABSTRACT

Compositions and methods for treating mucosa using compositions containing two or more peptides are described herein. The compositions can be used to treat various diseases associated with mucosa of the mouth, eye, ear, nose, esophagus, stomach, small intestinal, large intestine, rectum, vagina, urethra, penis, uterus, and the like and combinations thereof.

### B. CROSS-REFERENCE TO RELATED APPLICATIONS

This application claims priority from U.S. Provisional No. 62/979,898 entitled, “Peptides for Treating Mucosa,” filed Feb. 21, 2020, the entirety of which is hereby incorporated by reference.

### C. GOVERNMENT INTERESTS

Not applicable

### D. PARTIES TO A JOINT RESEARCH AGREEMENT

Not applicable

### E. INCORPORATION OF MATERIAL ON COMPACT DISC

Not applicable

### F. BACKGROUND

Not applicable

### G. SUMMARY OF THE INVENTION

Various embodiments of the invention include a composition for treating mucosa, including about 1 parts per million (ppm) to about 10 ppm tripeptide-1 and one or more dipeptide, tripeptide, tetrapeptide, or combinations thereof and about 1 ppm to about 10 ppm hexapeptide-12 and a hexapeptide with amino acid sequence different from the hexapeptide-12. In some embodiments, the composition is aqueous.

In some embodiments, the composition may include about 0.01 weight % to about 5.0 weight % phosphatidylserine based on the total weight of the composition. In various embodiments, the tripeptide-1 may be palmitoyl tripeptide-1, myristoyl tripeptide-1, or combinations thereof, and in some embodiments, the hexapeptide-12 may be palmitoyl hexapeptide-12, myristoyl hexapeptide-12, or combinations thereof. In certain embodiments, the composition may further include about 1 ppm to about 10 ppm tetrapeptide.

In some embodiments, the composition may further include one or more additional ingredients selected from the group consisting of disodium EDTA, niacinamide, caprylyl glycol, caprylhydroxamic acid, glycerin, phenoxyethanol, ethylhexylglycerin, betaine, propanediol, phospholipids, isopropyl palmitate, lecithin, polyacrylate-13, polysorbate 20, Squalane, Dunaliella Salina extract, phytosterols, Olea Europaea fruit oil, hydrolyzed pea protein, Butyrospermum Parkii (Shea) Butter, ceramide NP, tocopherol, butylene glycol, caprylyl methicone, ascorbyl palmitate, phosphatidylserine, and combinations thereof.

Other embodiments are directed to methods for treating mucosa, including the step of administering to a patient in need of treatment a composition including about 1 parts per million (ppm) to about 10 ppm tripeptide-1 and one or more dipeptide, tripeptide, tetrapeptide, or combinations thereof; and about 1 ppm to about 10 ppm hexapeptide-12 and a hexapeptide with amino acid sequence different from the hexapeptide-12.

In some embodiments, the composition may include about 0.01 weight % to about 5.0 weight % phosphatidylserine based on the total weight of the composition. In various embodiments, the tripeptide-1 may be palmitoyl tripeptide-1, myristoyl tripeptide-1, or combinations thereof, and in some embodiments, the hexapeptide-12 may be palmitoyl hexapeptide-12, myristoyl hexapeptide-12, or combinations thereof. In certain embodiments, the composition may further include about 1 ppm to about 10 ppm tetrapeptide.

In some embodiments, the composition may further include one or more additional ingredients selected from the group consisting of disodium EDTA, niacinamide, caprylyl glycol, caprylhydroxamic acid, glycerin, phenoxyethanol, ethylhexylglycerin, betaine, propanediol, phospholipids, isopropyl palmitate, lecithin, polyacrylate-13, polysorbate 20, Squalane, Dunaliella Salina extract, phytosterols, Olea Europaea fruit oil, hydrolyzed pea protein, Butyrospermum Parkii (Shea) Butter, ceramide NP, tocopherol, butylene glycol, caprylyl methicone, ascorbyl palmitate, phosphatidylserine, and combinations thereof.

In some embodiments, the mucosa may be oral mucosa, and the patient may be in need of treatment for gingivitis, mucosal lesions, periodontal disease, gum stomatitis, oral ulcers, oral candidiasis, oral lichen planus, mucous membrane pemphigoid, mucosal pemphigus vulgaris, chronic aphthous stomatitis, glossitis, oral herpes, cold sores, lesions or ulcers in the oral cavity, and combinations thereof.

In some embodiments, the mucosa may be nasal mucosa, and the patient may be in need of treatment for allergic rhinitis, hay fever, vasomotor rhinitis, non-allergic eosinophilic rhinitis, chronic sinusitis, inflammation of the paranasal sinuses, nasal polyps, eczematous nasal vestibulitis, epistaxis, inflammatory diseases associated with the nasal cavity, and combinations thereof.

In some embodiments, the mucosa may be vaginal mucosa, and the patient may be in need for treatment of atrophic vaginitis, cervical ectropia, follicular vulvitis, erythematous vulvitis, radiation-related vulvitis, genital herpes, increase vaginal lubrication, improve sexual arousal, and maintain elasticity of vaginal tissues after menopause, and combinations thereof.

In some embodiments, the mucosa may be rectal mucosa, and the patient may be in need of treatment for rectal pruritus, faecal incontinence, ulcerative proctitis, rectal prolapse, genital herpes, and combinations thereof.

In some embodiments, the mucosa may be ocular mucosa, and the patient may be in need of treatment for conjunctiva, keratoconjunctivitis sicca or anaphylaxis conjunctivitis, fungal infection, viral infection, bacterial infection, and combinations thereof.

In some embodiments, the mucosa may be aural mucosa, and the patient may be in need of treatment for earache, otitis media, otitis externa, ear infections, chronic otitis, meniere's disease, tinnitus, cerumen impaction, acoustic neuroma, mastoiditis, benign paroxysmal positional vertigo (BPPV), cholesteatoma, and combinations thereof.

In some embodiments, the mucosa may be intestinal mucosa, and the patient may be in need of treatment for Crohn's disease, ulcerative colitis, irritable bowel syndrome, celiac disease, herpetic dermatitis.

### H. DESCRIPTION OF THE DRAWINGS

Not applicable

### I. DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 ml to 8 ml is stated, 2 ml, 3 ml, 4 ml, 5 ml, 6 ml, and 7 ml are also intended to be explicitly disclosed, as well as the range of values greater than or equal to 1 ml and the range of values less than or equal to 8 ml.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition, or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The term “disorder” is used in this disclosure to mean, and is used interchangeably with the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance, reduce, normalize, or adjust the growth, texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc, which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government or listed in the U.S. Pharmacopeia or other generally recognized pharmacopeia for use in mammals (e.g. animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The following nomenclature is used to refer to amino acids: Alanine (“Ala” or “A”), Arginine (“Arg” or “R”), Asparagine (“Asn” or “N”), Aspartic acid (“Asp” or “D”), Cysteine (“Cys” or “C”), Glutamic acid (“Glu” or “E”), Glutamine (“Gln” or “Q”), Glycine (“Gly” or “G”), Histidine (“His” or “H”), Isoleucine (“Ile” or “I”), Leucine (“Leu” or “L”), Lysine (“Lys” or “K”), Methionine (“Met” or “M”), Phenylalanine (“Phe” or “F”), Proline (“Pro” or “P”), Serine (“Ser” or “S”), Threonine (“Thr” or “T”), Tryptophan (“Trp” or “W”), Tyrosine (“Tyr” or “Y”), and Valine (“Val” or “V”).

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is an adult or child human.

The term “treating” is used herein, for instance, in reference to methods of treating a disorder or a condition, and generally includes the administration of a compound or composition which reduces the frequency of, or delays the onset of, symptoms of a medical condition or enhance, reduce, normalize or adjust the growth, texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition. For example, in the context of a bacterial, microbial, fungal, or protozoal infection, “treating” refers to the reduction in bacterial, microbial, fungal, or protozoal load and/or improvement in symptoms related to the infection.

As used herein, the term “therapeutic” means an agent utilized to treat, combat, ameliorate, prevent or improve an unwanted condition or disease of a subject. In part, embodiments described herein may be directed to the treatment of various skin diseases, conditions, or disorders or symptoms thereof, including, but not limited to, benign proliferations, neoplasms, superficial blood vessel anomalies (tumors and malformations), epidermolysis bullosa, wounds and sores, Langerhans Cell Histiocytosis, Tuberous sclerosis, premalignancies, or malignancies of the skin, as well as the enrichment of immune cells in the skin. The skin condition may be a virally induced or non-virally induced cutaneous growth or proliferation. The skin condition may be an inflammatory condition. The skin condition may be a hyperproliferative condition. The skin condition may be a genetically-determined condition. The skin condition may be ageing including intrinsic and extrinsic changes (e.g., photoaging (ultraviolet light induced changes)), pigmentary changes, fine lines and rhytides. In some embodiments, the skin condition may be selected from Human Papilloma Virus induced lesions e.g., warts, common warts, palmoplantar warts, flat warts, recurrent warts, recalcitrant warts, treatment naïve warts, epidermodysplasia verruciformis related warts, anogenital warts, condyloma accuminatum, cervical dysplasias or neoplasias, e.g., cervical intraepithelial neoplasia (CIN); Herpesvirus related lesions including those induced by HHV-1 (HSV-1), HHV-2 (HSV-2), HHV-3 (varicella-zoster virus) e.g., chicken pox, Herpes zoster, shingles; Poxvirus induced lesions e.g., molluscum contagiosum, orf; callus, cutaneous horns, corns, acrochordons, fibroepithelial polyps, prurigo nodularis, actinic keratoses, squamous cell carcinoma, squamous cell carcinoma in situ, keratoacanthoma, basal cell carcinoma, cutaneous lymphomas and benign lymphocytic infiltrates & hyperplasias of the skin, clear cell acanthoma, large cell acanthoma, epidermolytic acanthoma, porokeratosis, hyperkeratosis, keratosis pilaris, lichenoid keratosis, acanthosis, acanthosis nigricans, confluent and reticulated papillomatosis, nevi, including e.g., dermal nevi, epidermal nevi, compound nevi, ILVEN (inflammatory linear verrucous epidermal nevi), nevus sebaceous, nevus comedonicus, and the like; acne, e.g., comedonal acne, inflammatory acne, papular acne, pustular acne, cystic acne; cysts, e.g., epidermoid cysts, milia, trichilemmal cysts, follicular cysts, proliferating cysts, dermoid cysts, pilonidal cysts, apocrine cysts, eccrine cysts, sebaceous cysts, mucous cysts, myxoid cysts, ganglion cysts, synovial cysts, vellus hair cysts, steatocystoma, hidrocystoma; adnexal neoplasms e.g., trichofolliculoma, fibrofolliculoma, perifollicular fibroma, trichodiscoma, nevus sebaceous, chondroid syringoma, trichoepithelioma, trichoblastoma, desmoplastic trichoepithelioma, pilomatricoma, pilomatrical carcinoma, tricholemmoma, trichelemmal carcinoma, tumor of the follicular infundibulum, tricoadenoma, proliferating pilar tumor, sebaceous hyperplasia, sebaceous adenoma, sebaceous epithelioma, sebaceous carcinoma, syringoma, poroma, hidradenoma, apocrine hidradenoma, spiradenoma, cylindroma, eccrine nevus (eccrine hamartoma), papillary adenoma, papillary adenocarcinoma; benign melanocytic proliferations or neoplasms e.g., ephilides, café-au-lait macules, Becker's melanosis, lentigines, solar lentigines, lentigo simplex, mucosal melanocytic lesions, Mongolian spots, Nevus of Ota, blue nevus, common acquired melanocytic nevi (nevocellular nevus, “moles”), congenital nevi, nevus spilus, recurrent nevi; vascular and perivascular neoplasms and reactive hyperplasias e.g., hemangiomas, cherry angiomas, hobnail hemangiomas (targeted hemosiderotic hemangiomas), tufted angiomas, hemangioendotheliomas, angiolymphoid hyperplasia with eosinophilia (ALHE), Glomus tumors (glomangiomas), hemangiopericytomas; cutaneous neural and neuroendocrine neoplasms e.g., neuromas, Schwannomas, neurofibromas, nerve sheath tumor, nerve sheath myxoma, neurothekeoma, granular cell tumor; fibrotic and fibrohistiocytic proliferations e.g., acrochordons, fibroepithelial polyps, fibromas, fibrous papules, angiofibromas, pearly penile papules, periungual fibromas, dermatofibromas, fibrokeratomas, sclerotic or pleomorphic fibromas, connective tissue nevi; cutaneous scars, hyperplasias, keloids, rosacea, cutaneous fungal, dermatophyte & mold infections, onychomycosis, hyperpigmentation, rhytides, psoriasis, malignant melanoma, seborrheic keratosis, seborrheic keratosis variants including e.g., dermatosis papulosis nigra, inverted follicular keratosis/keratoma warty dyskeratosis/warty dyskeratoma, acrokeratosis verruciformis, stucco keratosis; or a combination thereof.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed to compositions containing peptides formulated for delivery to mucosa, and methods for treating mucosa by administering such compositions to a subject in need of treatment. Mucosa is a membrane that lines various cavities in the body, consisting of one or more layers of epithelial cells overlying a layer of loose connective tissue. Mucosa is often continuous with the skin at various body openings such as the eyes, ears, inside the nose, inside the mouth, lips, vagina, urethral opening, and anus that secrete, for example, mucus, which binds to and stops pathogens and dirt from entering the body or prevents bodily tissues from becoming dehydrated.

In various embodiments, the compositions to may delivered to mucosa such as the mucosa in mouth, eye, ear, nose, esophagus, stomach, small intestinal, large intestine, rectum, vagina, urethra, penis, uterus, and the like and combinations thereof.

For example, in some embodiments, mucosa may be oral mucosa. Oral mucosa lines the mouth and includes tissues covering the roof of the mouth, gums, cheeks, lips, and throat. Diseases of the oral mucosa are common and often chronic. Such diseases include, for example, gingivitis, mucosal lesions, periodontal disease, gum stomatitis, oral ulcers, oral candidiasis, oral lichen planus, mucous membrane pemphigoid, mucosal pemphigus vulgaris, chronic aphthous stomatitis, glossitis, oral herpes, cold sores, and the like and combinations thereof, and gastrointestinal diseases that lead to inflammation, lesions, or ulcers in the oral cavity include, but are not limited to, Crohn's disease, ulcerative colitis, irritable bowel syndrome, celiac disease, and herpetic dermatitis. The compositions of various embodiments may improve the elasticity and resilience of oral mucosa improving the overall health of the tissue, alleviating the symptoms of the disease and improving healing.

In some embodiments, the mucosa may be nasal mucosa. Nasal mucosa lines the inside of the nose and sinuses and covers the periosteum and perichondrium of the nasal conchae. Diseases associated with nasal mucosa include, for example, allergic rhinitis, hay fever, vasomotor rhinitis, non-allergic eosinophilic rhinitis, chronic sinusitis, inflammation of the paranasal sinuses, nasal polyps, eczematous nasal vestibulitis, epistaxis, inflammatory diseases associated with the nasal cavity, and the like and combinations thereof. The compositions of various embodiments may improve the elasticity and resilience of nasal mucosa improving the overall health of the tissue, alleviating the symptoms of the disease and improving healing.

In some embodiments, the mucosa may be vaginal mucosa. Vaginal mucosa lines the vagina, extending from the vulva to the cervix. Diseases associated with the vagina include, for example, atrophic vaginitis, cervical ectropia, follicular vulvitis, erythematous vulvitis, radiation-related vulvitis, genital herpes, and the like and combinations thereof. In some embodiments, the compositions may be used to increase vaginal lubrication, improve sexual arousal, and maintain elasticity of vaginal tissues after menopause. The compositions of various embodiments may improve the elasticity and resilience of vaginal mucosa improving the overall health of the tissue, alleviating the symptoms of the disease and improving healing.

In some embodiments, the mucosa may be rectal mucosa. Rectal mucosa lines the rectum and extends from the rectosigmoidal junction to the anus. Rectal mucosa diseases include, for example, rectal pruritus, faecal incontinence, ulcerative proctitis, rectal prolapse, genital herpes, and the like and combinations thereof. The compositions of various embodiments may improve the elasticity and resilience of rectal mucosa improving the overall health of the tissue, alleviating the symptoms of the disease and improving healing.

In some embodiments, the mucosa may be the ocular mucosa, the conjunctiva. The ocular mucosa includes the bulbar at the anterior surface of the eye, which is transparent and avascular in the corneal region and loosely attached to the sclera, vascularised palpebral conjunctiva, which lines the posterior surface of the eyelids, and loose there are loose bridges of tissue between the bulbar and palpebral membrane, known as the superior and inferior fornices that form the conjunctival sac. Ocular mucosa protects the eye from foreign matter and continually washes the eye to remove foreign particles. Ocular mucosa further allows the eye to move within the eye socket and the eyelids to close over the eye. Non-limiting examples of diseases of the ocular mucosa that can be treated using the compositions of the invention include conjuntiva, keratoconjunctivitis sicca or anaphylaxis conjunctivitis, fungal infection, viral infection or bacteriological infection.

In some embodiments, the mucosa may be the mucosa of the ear, aural mucosa. Aural mucosa includes mucosa covering the external auditory canal, a short tube that ends at the eardrum (tympanic membrane) that secretes a waxy substance that inhibits bacterial growth and prevents epithelial maceration that can occur from residual moisture in the auditory canal, the middle ear that includes tiny bones that transfer vibrations from the tympanic membrane to the cochlea that transforms sound into nerve impulses and makes up the inner ear, fluid-filled semicircular canals (labyrinth) attached to the cochlea and nerves in the inner ear, and the eustachian (auditory) tube that drains fluid from the middle ear into the throat (pharynx) behind the nose. Aural mucosa covers the middle and inner ear canals as well as the auditory canal and eustachian tubes. Diseases that can be treated with the compositions of the invention include ear ache, otitis media (middle ear inflammation), otitis externa (Swimmer's ear), ear infections, chronic otitis, meniere's disease, tinnitus, cerumen (ear wax) impaction, acoustic neuroma, mastoiditis, benign paroxysmal positional vertigo (BPPV), cholesteatoma, and the like.

In some embodiments, the mucosa may be the mucosa of the intestines. Intestinal mucosa is the innermost layer of the gastrointestinal tract, surrounding the lumen and contacting digested food (chyme). Intestinal mucosa is made up of: Epithelium, the innermost layer, responsible for most digestive, absorptive and secretory processes; Lamina propria, a layer of connective tissue; Muscularis mucosae, a thin layer of smooth muscle that aids the passing of material and enhances the interaction between the epithelial layer and the contents of the lumen by agitation and peristalsis. The mucosae are highly specialized in each organ of the gastrointestinal tract to deal with the different conditions. The most variation is seen in the epithelium.

In various embodiments, the compositions may contain two or more peptides. In some embodiments, a first peptide may be a dipeptide such as, but are not limited to, KK, KP, CK, KC, KT, DF, NF, VW, YR, or TT. In some embodiments, a first peptide may be a tripeptide. including, but are not limited to, HGG, RKR, GHK, GKH, GGH, GHG, KFK, or KPK. In some embodiments, a first peptide may be a tetrapeptide such as, for example, GQPR, KTFK, AQTR, or RSRK. A second peptide in some embodiments may be a pentapeptide such as, but are not limited to, KTTKS, YGGFX or KLAAK. In some embodiments, a second peptide may be a hexapeptide such as, for example, VGVAPG or GKTTKS, and in some embodiments, a second peptide may be a heptapeptide including, but are not limited to, RGYYLLE, or Heptapeptide-6 (a pro-sirtuin peptide). The compositions of various embodiments, may include two or more peptides, for example, two dipeptides and one pentapeptide; one tripeptide and one hexapeptide; one dipeptide, one tripeptide, and one heptapeptide, or the like, provided that the composition contains at least one dipeptide, tripeptide, or tetrapeptide and at least one pentapeptide, hexapeptide, or heptapeptide.

In some embodiments, the weight ratio of first peptide to second peptide may be 1 part first peptide to about 0.2 to about 10 parts second peptide, about 1 to about 10, about 1 to about 8, about 1 to about 5, about 1 to about 1, about 0.5 to about 1, or any individual ratio encompassed by these examples.

In various embodiments, the composition may contain about 50 ppm to 50,0000 ppm first peptide, for example, about 50 ppm, 100 ppm, about 500 ppm, 1000 ppm, about 1500 ppm, about 5000 ppm, about 7500 pp, about 10,000 ppm, about 50,000 ppm, about 100,000 ppm or any range or individual concentration encompassed by these example concentrations. In some embodiments, the composition can contain from about 0.01 wt. % to about 10 wt. % first peptide based on the total weight of the composition, for example, about 0.01 wt. %, about 0.02 wt. %, about 0.03 wt. %, about 0.04 wt. %, about 0.05 wt. %, about 0.1 wt. %, about 1 wt. %, about 5 wt. %, about 10 wt. %, about 20 wt. % based on the total weight of the composition, or any individual concentration or range encompassed by these examples.

In various embodiments, the composition may contain about 50 ppm to 50,0000 ppm second peptide, for example, about 50 ppm, 100 ppm, about 500 ppm, 1000 ppm, about 1500 ppm, about 5000 ppm, about 7500 pp, about 10,000 ppm, about 50,000 ppm, about 100,000 ppm or any range or individual concentration encompassed by these example concentrations. In some embodiments, the composition can contain from about 0.01 wt. % to about 10 wt. % second peptide based on the total weight of the composition, for example, about 0.01 wt. %, about 0.02 wt. %, about 0.03 wt. %, about 0.04 wt. %, about 0.05 wt. %, about 0.1 wt. %, about 1 wt. %, about 5 wt. %, about 10 wt. %, about 20 wt. % based on the total weight of the composition, or any individual concentration or range encompassed by these examples.

Additional peptides other than the first peptide and the second peptide, i.e. a third peptide, a fourth peptide, a fifth peptide, and so on may be provided in the compositions within the same concentrations ranges described above for the first peptide and second peptide.

In some embodiments, the first peptide, second peptide, or additional peptides can be functionalized. For example, the peptide may include a covalently attached fatty acid, such as, for example, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, caprylic acid, capric acid, lauric acid, palmitic acid, stearic acid, arachidic acid, behenic acid, lignoceric acid, cerotic acid, or the like. In particular embodiments, the peptide may be functionalized with palmitoyl or myristoyl, which may provide enhanced peptide penetration of mucosa when compared to other fatty acids. Examples of functionalized peptides include, but are not limited to palmitoyl hexapeptide-12 (Pal-VGVAPG), palmitoyl tripeptide-1 (Pal-GHK), myristoyl hexapeptide-12 (Myr-VGVAPG), myristoyl tripeptide-1 (Myr-GHK).

In some embodiments, the first peptide may be glycine-histidine-lysine (GHK). GHK is a peptide sequence that is frequently found in extracellular matrix proteins, and its small size permits GHK to approach membrane receptors more easily than larger peptides. In some embodiments, GHK may be bound by copper (“GHK-Cu”). This copper-binding structure may enhance copper transport into and out of cells and promotes wound healing through several different but related pathways. GHK-Cu may act as an anti-inflammatory and antioxidant, and may promote wound healing by suppressing the “acute phase response” to the invasion of bacteria, facilitating aggregation of immune cells, stopping bleeding, providing a covering for the wounded area, and inhibiting the production of molecules called cytokines. GHK-Cu may also stimulate blood vessel growth, increase collagen production, and regenerate extracellular matrix, and may promote stem cell proliferation. By decreasing inflammation, acting as an antioxidant, stimulating growth of new blood vessels, regenerating the extracellular matrix, enhancing collagen production, and by promoting stem cell proliferation, GHK can enhance mucosal regeneration and promote mucosa healing.

Some embodiments the second peptide may be hexapeptide, valine-glycine-valine-alanine-proline-glycine (VGVAPG). VGVAPG is derived from the elastin protein and may promote skin regeneration by attracting monocytes and fibroblasts to injured mucosa facilitating regeneration of the extracellular matrix. VGVAPG may also provide a binding site for elastin-binding protein or a binding site for elastin and extracellular matrix degradation enzymes such as matrix metalloproteinases (MMPs), which facilitate the replacement and regeneration of elastic fibers and extracellular matrix proteins.

In certain embodiments, the amount of GHK or GHK-Cu in the compositions may be about 50 ppm to about 500 ppm, for example, about 50 ppm, about 100 ppm, about 150 ppm, about 200 ppm, about 300 ppm, about 400 ppm, about 500 ppm, or any range or individual concentration encompassed by these examples. The amount of VGVAPG in such embodiments may be about 50 ppm to about 500 ppm, for example, about 50 ppm, about 100 ppm, about 150 ppm, about 200 ppm, about 300 ppm, about 400 ppm, about 500 ppm, or any range or individual concentration encompassed by these examples.

In some embodiments, the compositions may include polyphenols such as oleuropein, a polyphenol isolated from olive leaves. Oleuropein may act as an anti-inflammatory agent, may enhance proteasome activity, decrease reactive oxygen species (ROS), reduce the amount of oxidized proteins, inhibit AGE formation, and the like and combinations thereof. The amount of polyphenol in such compositions may be about 0.005 wt. % to about 10 wt. % based on the total weight of the composition, about 0.01 wt. % to about 5.0 wt. %, about 0.05 wt. % to about 0.1 wt. %, or any range or individual concentration encompassed by these example ranges.

In some embodiments, the compositions may include phospholipids such as phosphatidylserine (PS), a membrane phospholipid component. Phosphatidylserine may activate signaling enzymes, act as an antioxidant, decrease metalloproteinases, increase procollagen formation, act as a substrate for AGE targets, reducing the damage from glycation effects, and the like. The amount of phospholipids in such compositions may be about 0.005 wt. % to about 10 wt. % based on the total weight of the composition, about 0.01 wt. % to about 5.0 wt. %, about 0.05 wt. % to about 0.1 wt. %, or any range or individual concentration encompassed by these example ranges.

In some embodiments, the compositions may include a mucoadhesive. Mucoadhesive may cover and protect the mucosa, preventing irritation and accelerating healing of inflamed or damaged tissue. Mucoadhesives may further promote long-term contact of the peptides with mucosal epithelium, enhancing activity. Mucoadhesives include, for example, poly (ethylene oxide), poly (ethylene glycol), poly (vinyl alcohol), poly (vinyl pyrrolidine), poly (acrylic acid), poly (hydroxyethyl methacrylate), hydroxyethyl ethyl cellulose, hydrogels containing about 0.05 wt. % to about 20 wt. % by total weight of the composition, water soluble polymer such as hydroxyethylcellulose, chitosan, and mixtures thereof, and the like and combinations thereof. In some embodiments, these compositions may also include a dispersing agent such as sodium carboxymethylcellulose.

Other useful compositions that promote mucoadhesion and retention of therapeutic agents for extended periods in the upper gastrointestinal tract are colloidal dispersions containing about 2 wt. % to about 50 wt. % colloidal particles such as silica or titanium dioxide. Such formulations are formed as mouthwash or as a low viscosity fluid liquid suitable for fine mist production. However, the particles interact with glycoproteins, particularly mucins, and the liquid turns into a viscous gel, providing effective mucoadhesion.

For oral administration and treatment of oral mucosa the compositions of embodiments may be formulated as an oral spray, rinse, emulsion, ointment, paste, gel, tablet, lozenge, or film.

Suitable spray delivery systems include both pressurized and non-pressurized (pump actuated) delivery means. The composition of such embodiments may be produced as a solution that may include, for example, emulsifiers, excipients, flavoring agents or odorants, preservatives and the like. In some embodiments, water may be the carrier, and in other embodiments, the composition may include a mixture of water-insoluble ingredients, such as propylene glycol, corn syrup, glycerin, sorbitol solutions, co-solvents, and the like and combinations thereof. Spray delivery systems typically deliver 50-100 μl in a single actuation and may require 1-5 actuations per dose. Patients may self-administer 1-5 times daily. The rheological properties of the spray formulation have been optimized to allow shearing and spraying for droplet formation. Furthermore, the spray delivery means are designed to produce droplets of a size that facilitates retention of the upper gastrointestinal tract on the mucosal surface and minimizes respiratory exposure.

In some embodiments, the compositions can be formulated as buccal rinses or mouthwashes that are prepared in a similar manner to the sprays described above, and may include, for example, emulsifiers, excipients, flavoring agents or odorants, preservatives and the like. In some embodiments, water may be the carrier, and in other embodiments, the composition may include a mixture of water-insoluble ingredients, such as propylene glycol, corn syrup, glycerin, sorbitol solutions, co-solvents, and the like and combinations thereof. Administration buccal rinses and mouthwashes can be carried out using this preparation by irrigating the oral cavity, or rinsing. In some embodiments, the composition may be swallowed, providing therapy to the esophagus, stomach, and intestine.

In certain embodiments, the peptides may be encapsulated in, for example, biodegradable microspheres rather than dissolved in the liquid phase of the formulation. Various microencapsulated drug delivery systems exist and can be used in such embodiments. Polymers commonly used to produce microspheres include, for example, poly-ε-caprolactone, poly (ε-caprolactone-Co-DL-lactic acid), poly (DL-lactic acid), poly (DL-lactic acid-Co-glycolic acid), and poly (ε-caprolactone-Co-glycolic acid). Microspheres can be made by techniques well known in the art, including spray drying, coacervation, and emulsification.

In some embodiments, the compositions may be formulated as an ointment, paste, or gel. Such formulations are highly viscous and can be applied directly to a wound, or included in, for example, toothpaste. Anionic polysaccharides such as pectin and gellan can be used to create a gel composition.

The toothpastes of various embodiments may contain an effective amount of the peptides described above as well as various ingredients known in the art and useful in toothpastes, including, for example, polishing agents, flavoring agents, sweeteners, bacteriocides, oxidizing agents, thickeners, carbonate and bicarbonate salts, and the like.

Examples of the polishing agent are calcium secondary phosphate dihydrate and anhydride, calcium primary phosphate, calcium tertiary phosphate, calcium carbonate, calcium pyrophosphate, aluminum hydroxide, alumina, insoluble sodium metaphosphate, magnesium tertiary phosphate, magnesium carbonate, calcium sulfate, polymethyl methacrylate, synthetic resins, and the like and combinations thereof. The amount of polishing agent in the compositions of embodiment may be about 5% to about 50% by weight, about 10% to about 40% by weight based on the total weight of the composition, or any range or individual concentration encompassed by these example ranges. In some embodiments, the compositions may include dicalcium phosphate, a metal of the carbonate or bicarbonate such as sodium, potassium, calcium, magnesium, aluminum, zinc, lithium, and the like and combinations thereof.

Humectants and wetting agents, useful in such embodiments, include deionized water, alone or combined with sorbitol, glycerine, ethylene glycol, propylene glycol, 1,3-butylene glycol, polypropylene glycol, polyethylene glycol, xylitol, maltitol, lactitol, or a copolymer of polypropylene and polyethylene glycol ranging in molecular weight from 200 to 1,000, and the like and combinations thereof. The amount of humectant/wetting agent used in the compositions may be about 5% to about 70% by weight based on the total weight of the composition, or any individual value or range encompassed by these ranges. The compositions may further include a thickening agent such as, for example, cornstarch, gelling agent, such as, cellulose gum, polysaccharides, guar gum, sodium carrageenan, calcium carrageenan, gum tragacanth, Karaya, hydrated silica, Xanthan gum, Carbopol 940, glycerides of starch, V-gum (magnesium aluminum silicate), and the like and combinations thereof. The amount of thickener used in the compositions may be about 5% to about 70% by weight based on the total weight of the composition, or any individual value or range encompassed by these ranges.

Flavoring agents include, but are not limited to, menthol, carvone, anethole, eugenol, methyl salicylate, limonene, cymene, n-decyl alcohol, citronellol, α-terpineol, methyl acetate, citronellyl acetate, methyl eugenol, cineole, linalool, ethyl linalool, vanillin, thymol, spearmint oil, peppermint oil, lemon oil, orange oil, sage oil, rosemary oil, cinnamon oil, pellira oil, gaultheria oil, clove oil, eucalyptus oil, and the like and combinations thereof. The amount of flavoring agent used in such embodiments can be about 0.1% to about 10% by weight, about 0.5% to 5% by weight based on the total weight of the composition, or any range or individual value encompassed by these example ranges. In some embodiments, the compositions may include a sweetener in place of or in combination with a flavoring agent. Sweeteners include, for example, saccharin sodium, Acesulfame K, stevioside, neo-hesperidyl dihydrochalcone, glycyrrhizin, perillartine, thaumatin, aspartylphenylalanine methyl ester, p-methoxycinnamic aldehyde, and the like and combinations thereof. The amount of sweetener used may be about 0.01% to about 1% by weight, about 0.05% to about 0.5% by weight based on the total weight of the composition, or any range or individual amount encompassed by these example ranges.

In some embodiments, the compositions may include an oxidizing agent such as, for example, calcium peroxide, zinc peroxide, sodium peroxide, sodium persulfate, sodium percarbonate or sodium perphosphate. In some embodiments, the compositions may include a detergent such as, for example, sodium lauryl sulfoacetate, sodium lauryl sulfate, and the like and combinations thereof. The pH of the compositions may be adjusted to a pH of about 3 and about 10 about using agents such as, for example, aluminum hydroxide, sodium carbonate, potassium hydroxide, or sodium hydroxide, or an organic acid such as citric acid, propionic acid or a mild weak dilution of an inorganic mineral acid such as hydrochloric acid.

In some embodiments, the toothpaste compositions may further include a bactericides such as, for example, tricrosan, amphoteric bactericides such as dodecyldiaminoethylglycine, enzymes such as dextranase, amylase, protease, mutanase, lysozyme and lytic enzymes, monofluorophosphates of alkali metals, such as sodium monofluorophosphate and potassium monofluorophophate, fluorides such as sodium fluoride and stannous fluoride, tranexarnic acid and ε-aminocapric acid, aluminum chlorhydroxyl allantoin, dihydrocholesterol, glycyrrhizin salts, glycyrrhetic acid, glycerophosphate, chlorophyll, sodium chloride, caropeptide, water-soluble compounds of inorganic phosphoric acid, and the like and combinations thereof.

In some embodiments, the peptides described above may be administered to oral mucosa using film, tablet, lozenge, gelcap, or liquid capsule, formulated to be solid at room temperature, melt at body temperature, and release the therapeutic agent. Polymeric films may stay in the oral cavity for extended periods of time (in hours to days), during which time continuous release occurs. Usually, the film is partially or fully biodegradable and includes a mucoadhesive layer to secure the film to the oral mucosa. In some embodiments, films means may further prevent mechanical damage or microbial infection of the lesion site. This physical barrier function is particularly beneficial for the treatment of conditions such as mucositis or aphthous stomatitis. Films may also release the peptides of the compositions above directly into the underlying mucosa, oral cavity, or combinations thereof.

In some embodiments, films may include two or more layers: a mucoadhesive layer suitable for adhering the film to the oral mucosa, and a body layer containing the active therapeutic agent. Many suitable mucoadhesives are well known in the art. In some embodiments, one or more therapeutic agents can also be provided in the adhesive layer. The body layer can include, for example, starch, gelatin, polyethylene glycol, polypropylene glycol, polyethylene oxide, copolymers of ethylene oxide and propylene oxide, copolymers of polyethylene glycol and polypropylene glycol, polytetramethylene glycol, polyether urethane, hydroxyethyl cellulose, ethyl cellulose, hydroxypropylcellulose, hydroxypropylmethylcellulose, alginate, collagen, polylactic acid, poly (lactic acid-co-glycolide) (PLGA), calcium polycarbophil, polyethyl methacrylate, cellulose acetate, propylene glycol, polyacrylic acid, crosslinked polyacrylic acid, hydroxyethyl methacrylate/methyl methacrylate copolymer, silicon/tillcellulose/polyethylene glycol, urethane polyacrylate, polystyrene, polysulfone, polycarbonate, polyorthoester, polyanhydride, poly(amino acid), partially or fully hydrolyzed alkylene-vinyl acetate copolymer, polyvinyl chloride, polyacetic acid, polymers of vinyl, polyvinyl alkyl esters, styrene acrylonitrile copolymers, poly (ethylene terephthalate), polyalkylenes, poly (vinyl imidazole), polyesters, and the like and combinations thereof. In particular embodiments, the body layer polymer may include PLGA and ethylcellulose.

In some embodiments, the peptide compositions described above may be contained in tablets, lozenges, or chewing gum. Such formulations are well known in the art and generally include a sugar, starch, or lipid, and a flavoring base. Tablets and lozenges can be prepared by art-recognized techniques for forming compressed tablets where the peptides of the invention are dispersed on a compressible solid carrier, optionally combined with any appropriate tableting aids such as a lubricant (e.g., magnesium-stearate) and is compressed into tablets. The solid carrier component for such tableting formulations can be a saliva-soluble solid, such as a cold water-soluble starch or a monosaccharide, so that the lozenge will readily dissolve in the mouth to release the contained disaccharide acid in saliva solution for contact with and absorption by the oral/pharyngeal mucosa when the lozenge is held in the mouth. The pH of the above-described formulations can range from about 4 to about 8.5.

In certain embodiments, oral mucosa may be treated using a balm including the peptide compositions described above. Such balms may be applied to, for example, lips, tongue, cheeks, and the like using an applicator or as a semisolid stick. Balms can be prepared by any means known in the art and may contain various ingredients described below in relation to topical compositions.

In embodiments in which the compositions are designed to be applied topically to nasal mucosa, vaginal mucosa, or rectal mucosa, the compositions of various embodiments may include a base such as, for example, white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, PCCA plasticized base, and the like and combinations thereof.

In some embodiments, the base may be a liposomal base. Liposomal bases are an emulsion that includes a lipophilic component and an aqueous component that can be in the form of a lotion, a cream, a gel, or a paste. Examples of suitable liposomal bases include PCCA Lipoderm®, Lipoderm ActiveMax™, Anhydrous Lipoderm, and Lipoderm High Molecular Weight™ PCCA. Such liposomal base formulations can include, for example, about 60-80% wt/wt water combined with glycerin, C₁₂₋₁₅ alkyl benzoate, glyceryl stearate, dimethicone, cetearyl alcohol, cetearyl glucoside, polyacrylamide, cetyl alcohol, magnesium aluminum silicate, xanthan gum, aloe vera (aloe barbadensis), tocopheryl acetate (vitamin E acetate), prunus amygdalus amara (bitter almond) kernel oil, vitis vinifera (Grape) seed extract, triticum vulgare (wheat) germ oil, retinyl palmitate (vitamin A palmitate), ascorbyl palmitate (vitamin C palmitate), Pro-Lipo Multi-emulsion Liposomic System, tetrasodium EDTA, phenoxyethanol, sodium hydroxymethylglycinate and the like and combinations thereof.

In some embodiments, the base may be cream base. Cream bases are semi-solid emulsions of oil and water. They are divided into two types: oil-in-water (O/W) creams which are composed of small droplets of oil dispersed in a continuous water phase, and water-in-oil (W/O) creams which are composed of small droplets of water dispersed in a continuous oily phase. Oil-in-water creams are more comfortable and cosmetically acceptable as they are less greasy and more easily washed off using water. Water-in-oil creams are more difficult to handle but many drugs which are incorporated into creams are hydrophobic and will be released more readily from a water-in-oil cream than an oil-in-water cream. Water-in-oil creams are also more moisturising as they provide an oily barrier which reduces water loss from the stratum corneum, the outermost layer of the skin. Cream bases typically include water, oil, emulsifier, and thickening agents, such as those discussed below.

In some embodiments, the base may be a moisturizing cream base. Moisturizing cream bases are composed of the same components as the cream bases described above with the addition of an emollient or humectant, that may provide a barrier that reduces water loss from the stratum corneum, the outermost layer of the skin. The emollient or humectant in a moisturizing cream base may be cetyl esters wax, stearyl alcohol, cetyl alcohol, and glycerin, or combinations thereof. Example cream bases and moisturizing cream bases include VersaBase (PCCA); Emollient cream, Vanishing cream, CeraVe, Vanicream, Vitamin E; Cliniderm; Dermabase (purified water, petrolatum, mineral oil, cetostearyl alcohol); Eucerin (water, petrolatum, mineral oil, ceresin, lanolin alcohol, methylchloroisothiazolinone, methylisothiazolinone); Glaxal (WellSpring Pharmaceutical Corp., Sarasota, Fla.); stearic acid cream, or any other pharmaceutical cream base used for topical formulations known to those skilled in the art.

In some embodiments, the base may be an ointment base. Ointments are compositions in which oil and water are provided in a ratio of from 7:1 to 2:1, from 5:1 to 3:1, or 4:1, and in some embodiments, the ointment may or may not include water, such as Aquaphor, Pracasil, and plasticized bases. Ointments are generally formulated using oils, waxes, water, alcohols, petroleum products, silicones, water, and other agents to prepare formulations with various viscosities and solvent properties. Commonly used formulations include oleaginous base (White Ointment), absorption base, W/O emulsion base (Cold Cream type base), O/W emulsion base (Hydrophilic Ointment), water soluble base, in addition to others. These preparations are used to dissolve or suspend substances or products with medicinal or cosmetic value.

In some embodiments, the base may be an emollient base. Non-limiting examples of emollient bases includes C₉-C₁₄ linear or branched alkyl alcohols, C₃-C₁₄ linear or branched polyols, C₆-C₁₄ di-esters of C₆-C₁₂ diacids, hydrocarbons, natural waxes, vegetable oils, and silicones, branched chain esters, ethoxylated partial glyceride fatty acid esters, protein derivatives, lanolin and lanolin derivatives, and fatty alcohol ethoxylates, emollient oils, fatty acids, fatty alcohols and their esters. such as, for example, isononyl isonanoate, dioctyl sebacate, isooctyl isooctanoate, dioctyl adipate, squalane, petrolatum, mineral oil, carnauba wax, candelilla wax, beeswax, sunflower oil, sesame oil, olive oil, cyclomethicone and dimethicone.

In some embodiments, the emollient base may be or may include polyols having the formula:

HOCH₂—[CHOH]ₓ—CH₂OH

wherein the index x is an integer from 1 to 20. In some embodiments, x is an integer from 1 to 10. Examples, of such polyols include glycerol, erythritol, xylitol, (2R,3R)-butane-1,2,3,4-tetrol, (2S,3R)-butane-1,2,3,4-tetraol, (2R,3S)-butane-1,2,3,4-tetraol, (2S,35)-butane-1,2,3,4-tetrol, (2R,3R,4R)-pentane-1,2,3,4,5-pentol, (2S,3R,4R)-pentane-1,2,3,4,5-pentol, (2R,3S,4R)-pentane-1,2,3,4,5-pentol, (2R,3R,4S)-pentane-1,2,3,4,5-pentol, (2S,3S,4R)-pentane-1,2,3,4,5-pentol, (2S,3R,4S)-pentane-1,2,3,4,5-pentol, (2R,3S,4S)-pentane-1,2,3,4,5-pentol, and (2S,3S,4S)-pentane-1,2,3,4,5-pentol. In some embodiments, the emollient base may be glycerol.

The amount of base in the compositions of embodiments can vary and will depend on the amounts of the other components. More base can be added to compensate for smaller amounts of other components in the desired topical pharmaceutical formulation. In some embodiments, the base may be present in a concentration of about 45% (w/w) to about 99.75% (w/w) of the total composition, or any range or individual concentration known in the art.

In some embodiments, the compositions may further include a solubility enhancer. A solubility enhancer may be necessary to produce a topical composition that effectively delivers the aminothiols, biguanides, and combinations thereof, to affected tissues. The solubility enhancers are not limited and may include various known solubility enhancers and combinations thereof. In particular embodiments, the solubility enhancer may be, for example, ethyl acetate, ethanol, methanol, dimethylformamide (DMF), acetone, acetonitrile, tetrahydrofuran (THF), acetic acid, dimethyl sulfoxide (DMSO), chloroform, propylene glycol, polyethylene glycol, propane-1,3-diol and the like and combinations thereof, and in some embodiments, the solubility enhancer may be DMSO. The composition may include about 10% (w/w) to about 40% (w/w) solubility enhancer, and in some embodiments, the composition may include at least about 25% (w/w) to about 35% (w/w) solubility enhancer.

The compositions of various embodiments can be in any form, including, for example, liquid, creams, lotions, foams, liniments, and the like.

In some embodiments, the compositions described above may be formulated as a liquid. Liquid dosage forms for topical administration may include diluents such as, for example, alcohols, glycols, oils, water, and the like. Such compositions may also include wetting agents or emulsifiers. Such liquid compositions may be particularly well suited for nasal mucosa administration as, for example, a nasal spray, eye drops for treating ocular mucosa, and ear drops for treating aural mucosa. In other embodiments, liquid dosages can be used to treat oral mucosa when formulated as an oral rinse or mouthwash.

In some embodiments, the compositions of embodiments may be formulated as oil-in-water or water-in-oil emulsion. A cream can be a water-in-oil (w/o) emulsion in which an aqueous phase is dispersed in an oil phase, or an oil-in-water (o/w) emulsion in which an oil is dispersed within an aqueous base. An ointment generally refers to a more viscous oil-in-water cream. Traditional ointment bases (i.e. carrier) include hydrocarbons (petrolatum, beeswax, etc.) vegetable oils, fatty alcohols (cholesterol, lanolin, wool alcohol, stearyl alcohol, etc.) or silicones. Insoluble solids such as starch, zinc oxide, calcium carbonate, or talc can also be used in ointments and creams. Gel forms of the compositions described above can be formed by the entrapment of large amounts of aqueous or aqueous-alcoholic liquids in a network of polymers or of colloidal solid particles. Such polymers or colloids (gelling or thickening agents) are typically present at concentrations of less than 10% w/w and include carboxymethyl cellulose, hydroxypropylmethyl cellulose, hydroxyethyl cellulose, methyl cellulose, sodium alginate, alginic acid, pectin, tragacanth, carrageen, agar, clays, aluminum silicate, carbomers, and the like.

In some embodiments, the compositions described above may further include one or more pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, and the like and combinations thereof. The person of ordinary skill in the art can refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, New York (1980) for guidance in determining the amount of such components in the compositions and formulations of embodiments.

In some embodiments, the topical formulations can be in the form of a lotion. Lotions are low- to medium-viscosity topical preparation. Most lotions are oil-in-water emulsions containing an emulsifier such as cetyl alcohol to prevent separation of these two phases. Lotions can include fragrances, glycerol, petroleum jelly, dyes, preservatives, proteins and stabilizing agents.

In some embodiments, the topical formulations can be in the form of a foam. Pharmaceutical foams are pressurized dosage forms containing one or more active ingredients that, upon valve actuation, emit a fine dispersion of liquid and/or solid materials in a gaseous medium. Foam formulations are generally easier to apply, are less dense, and spread more easily than other topical dosage forms. Foams may be formulated in various ways to provide emollient or drying functions to the skin, depending on the formulation constituents. Accordingly, this delivery technology is a useful addition to the spectrum of formulations available for topical use.

In some embodiments, the topical formulations can be in the form of a liniment. Liniments or balms are topical formulations that are of a similar viscosity to lotions and less viscous than an ointment or cream. Liniments are generally applied with friction by rubbing the liniment into the skin. Liniments typically are formulated from alcohol, acetone, or similar quickly evaporating solvents and may contain counterirritant aromatic chemical compounds such as methyl salicylate, benzoin resin, or capsaicin.

Emollient or lubricating vehicles that help hydrate the skin can also be used. Examples of suitable bases or vehicles for preparing hydrating compositions for use with human skin are petrolatum, petrolatum plus volatile silicones, lanolin, cold cream (USP), and hydrophilic ointment (USP).

Vitamins include, for example, vitamin D, vitamin K, vitamin B (including niacinamide, nicotinic acid, C1-18 nicotinic acid esters, and nicotinyl alcohol; B6 compounds, such as pyroxidine; and B5 compounds, such as panthenol, or “pro-B5”), vitamin A (including retinoids such as retinyl propionate, carotenoids, and other compounds), vitamin E (including tocopherol sorbate, tocopherol acetate, other esters of tocopherol), vitamin C (including ascorbyl esters of fatty acids, and ascorbic acid derivatives, for example, ascorbyl glucoside, magnesium ascorbyl phosphate, sodium ascorbyl phosphate, and ascorbyl sorbate), and all natural and/or synthetic analogs thereof, and combinations thereof. In various embodiments, the compositions may include about 0.0001 wt. % to about 50 wt. %, about 0.001 wt. % to about 10 wt. %, about 0.01 wt. % to about 5 wt. %, or about 0.1 wt. % to about 1 wt. %, or any individual concentration or range of each vitamin contained in the composition.

In some embodiments, the compositions may include an antioxidant. Such antioxidant may be, for example, butylated hydroxytoluene, ascorbic acid, ascorbic palmitate, butylated hydroxyani sole, 2,4,5 -trihydroxy butyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant can be present in a concentration of about 0.01% (w/w) to about 1% (w/w) of the total composition or any individual concentration encompassed by this example range.

In some embodiments, the composition may include an emulsifying agent including, for example, various monoglycerides, diglycerides, triglycerides, and blends thereof at a concentration of about 3% (w/w) to about 10% (w/w) of the total composition.

In some embodiments, the compositions may further include a humectant that provides soothing, smoothing, moisturizing, or protects the skin. The humectant is not limited and can be, for example, calamine, dodecyl sulphate, sodium lauryl sulphate (SLS), a polyoxyethylene ester of polysorbitan, such as monooleate, monolaurate, monopalmitate, monostearate esters, esters of sorbitan, the polyoxyethylenes ethers, the sodium dioctyl sulfosuccinate (DOSS), lecithin, and sodium docusate. The amount of humectant in such compositions may be about 0.01% (w/w) to 5% (w/w) of the total composition.

In some embodiments, the composition may further include an analgesic agent such as, for example, methyl salicylate, codeine, morphine, methadone, pethidine, buprenorphine, hydromorphone, levorphanol, oxycodone, fentanyl, a non-steroidal anti-inflammatory drug (NSAID), and the like and combinations thereof. The amount of the analgesic agent such compositions may be about 0.01% (w/w) to 5% (w/w) of the total composition.

In some embodiments, the compositions may further include a topical debriding agent such as, for example, papain/urea, balsam peru/castor oil/trypsin, chlorophyllin copper complex/papain/urea, collagenase, and the like and combinations thereof. The amount of the debriding agent in such compositions may be about 0.01% (w/w) to 5% (w/w) of the total composition.

In some embodiments, the compositions described above can be formulated for oral administration to treat diseases of the intestinal mucosa such as, for example, food allergies, inflammatory bowel diseases, irritable bowel syndrome, ulcerative colitis, Crohn's disease, celiac disease, metabolic syndrome, non-alcoholic fatty liver disease, diabetes, septic shock, and the like and combinations thereof.

In certain embodiments, compositions for oral delivery may be formulated as controlled release drug delivery systems including the composition including peptides described above and a polymeric matrix that degrades in the gastrointestinal tract. In some embodiments, the formulation may include a hydrogel-forming polymer such as acrylic acid derivatives, polycarbophil, Carbopol, cellulose derivatives, for example, methylcellulose, carboxymethyl cellulose, and hydroxypropyl cellulose, acrylic acid or acrylic acid derivatives; for example methylmethacrylate, natural polymer, for example, guar gum, acacia gum, agar, tragacanth, alginic acid, dextran, arabinogalactan, pectin, egg albumin, soybean protein, and hyaluronic acid, modified natural polymer, for example, calcium pectinate, calcium alginate, modified egg albumin or modified soybean protein, and the like and combinations thereof. In some embodiments, the hydrogel-forming polymer may be a blend of polymers (for example, a blend of polycarbophil and Eudragit® RL-100).

Such compositions can be in the form of tablets, capsules, gel caps, and the like, and may include various additional components such as pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, and the like and combinations thereof. The person of ordinary skill in the art can refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, New York (1980) for guidance in determining the amount of such components in the compositions and formulations of embodiments.

Other embodiments of the invention include methods for treating mucosa by administering any of the compositions described above. The methods of various embodiments may include the steps of administering a composition of the various embodiments described above to affected mucosa of a subject in need of treatment. The step of administering can be carried out by various means. For example, administering can be accomplished by applying the composition to the mucosa of a subject, and in some embodiments, the mucosa may be massaged or rubbed to facilitate contacting the affected area. In other embodiments, administering can be carried out by administering a tablet, lozenge, or film to the mouth of a patient in need of treatment and allowing the tablet, lozenge, or film to dissolve, and in some embodiments, the compositions of the invention may be administered by spraying the composition directly onto the mucosa of, for example, the mouth or nasal passages.

The step of administering can be carried out one, two, three, four, or more times per day, and administering can be carried out the prescribed number of times per day for one week to six months or until the symptoms are resolved. In some embodiments, improvement in one or more symptoms may be observed within about 7 days of treatment, and in certain embodiments, improvement in one or more symptoms may be observed within about 1, about 2, about 3, about 4, about 5, or about 6 days after initial treatment.

Further embodiments include methods for making the compositions containing the peptide compositions described above. Such embodiments may generally include the step of contacting the peptide compositions with a solubility enhancer or solvent to create a peptide containing solution and combining the peptide containing solution with a base. The base may be any of the bases described above used to create, for example, topical liquids, creams, lotions, foams, liniments, and the like. As discussed above, the solubility enhancer may be, for example, ethyl acetate, ethanol, methanol, dimethylformamide (DMF), acetone, acetonitrile, tetrahydrofuran (THF), acetic acid, dimethyl sulfoxide (DMSO), chloroform, propylene glycol, polyethylene glycol, propane-1,3-diol and the like and combinations thereof, and in some embodiments, the solubility enhancer may be DMSO.

In some embodiments, the base may be a cream base. Cream bases can be prepared separately by conventional techniques well known to those skilled in the art. Generally, a suitable process includes admixing the various ingredients of the cream in appropriate relative amounts in any order that is convenient and thereafter, if necessary, adjusting the pH to the final desired value. For example, the components of the base may be mixed together at a temperature of about 65° C. to about 75° C. until an emulsion has formed, and therapeutic agent may be added after cooling the emulsified cream base or during mixing.

As is known in the art, certain means for administering may require the use of particular components of the formulation. Such components are described above and can be appropriately incorporated into the compositions.

1. A composition for treating mucosa, comprising:
about 1 parts per million (ppm) to about 10 ppm tripeptide-1 and one or more dipeptide, tripeptide, tetrapeptide, or combinations thereof; and
about 1 ppm to about 10 ppm hexapeptide-12 and a hexapeptide with amino acid sequence different from the hexapeptide-12.

2. The composition of claim 1, wherein the composition is aqueous.

3. The composition of claim 1, further comprising one or more additional ingredients selected from the group consisting of disodium EDTA, niacinamide, caprylyl glycol, caprylhydroxamic acid, glycerin, phenoxyethanol, ethylhexylglycerin, betaine, propanediol, phospholipids, isopropyl palmitate, lecithin, polyacrylate-13, polysorbate 20, Squalane, Dunaliella Salina extract, phytosterols, Olea Europaea fruit oil, hydrolyzed pea protein, Butyrospermum Parkii (Shea) Butter, ceramide NP, tocopherol, butylene glycol, caprylyl methicone, ascorbyl palmitate, phosphatidylserine, and combinations thereof.

4. The composition of claim 1, further comprising about 0.01 weight % to about 5.0 weight % phosphatidylserine based on the total weight of the composition.

5. The composition of claim 1, wherein the tripeptide-1 is selected from the group consisting of palmitoyl tripeptide-1, myristoyl tripeptide-1, and combinations thereof.

6. The composition of claim 1, wherein the hexapeptide-12 is selected from the group consisting of palmitoyl hexapeptide-12, myristoyl hexapeptide-12, and combinations thereof.

7. The composition of claim 1, further comprising about 1 ppm to about 10 ppm tetrapeptide.

8. A method for treating mucosa, comprising administering to a patient in need of treatment a composition including:
about 1 parts per million (ppm) to about 10 ppm tripeptide-1 and one or more dipeptide, tripeptide, tetrapeptide, or combinations thereof; and
about 1 ppm to about 10 ppm hexapeptide-12 and a hexapeptide with amino acid sequence different from the hexapeptide-12.

9. The method of claim 8, wherein the composition further comprises about 0.01 weight % to about 5.0 weight % phosphatidylserine based on the total weight of the composition.

10. The method of claim 8, wherein the tripeptide-1 is selected from the group consisting of palmitoyl tripeptide-1, myristoyl tripeptide-1, and combinations thereof.

11. The method of claim 8, wherein the hexapeptide-12 is selected from the group consisting of palmitoyl hexapeptide-12, myristoyl hexapeptide-12, and combinations thereof.

12. The method of claim 8, further comprising about 1 ppm to about 10 ppm tetrapeptide.

13. The method of claim 8, wherein the composition further comprises one or more additional ingredients selected from the group consisting of disodium EDTA, niacinamide, caprylyl glycol, caprylhydroxamic acid, glycerin, phenoxyethanol, ethylhexylglycerin, betaine, propanediol, phospholipids, isopropyl palmitate, lecithin, polyacrylate-13, polysorbate 20, Squalane, Dunaliella Salina extract, phytosterols, Olea Europaea fruit oil, hydrolyzed pea protein, Butyrospermum Parkii (Shea) Butter, ceramide NP, tocopherol, butylene glycol, caprylyl methicone, ascorbyl palmitate, phosphatidylserine, and combinations thereof.

14. The method of claim 1, wherein the mucosa is oral mucosa and the patient is in need of treatment for gingivitis, mucosal lesions, periodontal disease, gum stomatitis, oral ulcers, oral candidiasis, oral lichen planus, mucous membrane pemphigoid, mucosal pemphigus vulgaris, chronic aphthous stomatitis, glossitis, oral herpes, cold sores, lesions or ulcers in the oral cavity, and combinations thereof.

15. The method of claim 1, wherein the mucosa is nasal mucosa and the patient is in need of treatment for allergic rhinitis, hay fever, vasomotor rhinitis, non-allergic eosinophilic rhinitis, chronic sinusitis, inflammation of the paranasal sinuses, nasal polyps, eczematous nasal vestibulitis, epistaxis, inflammatory diseases associated with the nasal cavity, and combinations thereof.

16. The method of claim 1, wherein the mucosa is vaginal mucosa and the patient is in need for treatment of atrophic vaginitis, cervical ectropia, follicular vulvitis, erythematous vulvitis, radiation-related vulvitis, genital herpes, increase vaginal lubrication, improve sexual arousal, and maintain elasticity of vaginal tissues after menopause, and combinations thereof.

17. The method of claim 1, wherein the mucosa is rectal mucosa and the patient is in need of treatment for rectal pruritus, faecal incontinence, ulcerative proctitis, rectal prolapse, genital herpes, and combinations thereof.

18. The method of claim 1, wherein the mucosa is ocular mucosa, and the patient is in need of treatment for conjunctiva, keratoconjunctivitis sicca or anaphylaxis conjunctivitis, fungal infection, viral infection, bacterial infection, and combinations thereof.

19. The method of claim 1, wherein the mucosa is aural mucosa, and the patient is in need of treatment for earache, otitis media, otitis externa, ear infections, chronic otitis, meniere's disease, tinnitus, cerumen impaction, acoustic neuroma, mastoiditis, benign paroxysmal positional vertigo (BPPV), cholesteatoma, and combinations thereof.

20. The method of claim 1, wherein the mucosa is intestinal mucosa, and the patient is in need of treatment for Crohn's disease, ulcerative colitis, irritable bowel syndrome, celiac disease, herpetic dermatitis.
